(19) United States
(12) Patent Application Publication
(10) Pub. No.: US 2017/0227532 A1
(43) Pub. Date: Aug. 10, 2017

(54) METHOD FOR PRODUCING A CAPTURE PHASE FOR THE DETECTION OF A BIOLOGICAL TARGET, AND ASSOCIATED DETECTION METHODS AND KITS

(71) Applicants: BIOMERIEUX, Marcy L'Etoile (FR); UNIVERSITE D'AIX MARSEILLE, Marseille Cedex 07 (FR); CENTRE NATIONAL DE LA RECHERCHE SCIENTIFIQUE, Paris Cedex 16 (FR)

(72) Inventors: Laure ALLARD, Craponne (FR); Thomas TRIMAILLE, Craponne (FR)

(73) Assignees: BIOMERIEUX, Marcy L'Etoile (FR); UNIVERSITE D'AIX MARSEILLE, Marseille Cedex 07 (FR); CENTRE NATIONAL DE LA RECHERCHE SCIENTIFIQUE, Paris Cedex 16 (FR)

(21) Appl. No.: 15/501,714

(22) PCT Filed: Aug. 3, 2015
(86) PCT No.: PCT/FR2015/052146

§ 371 Date: Feb. 3, 2017

(30) Foreign Application Priority Data
Aug. 4, 2014 (FR) 1457578

(51) Int. Cl. G01N 33/543; G01N 33/545; G01N 33/531

(52) U.S. Cl. CPC G01N 33/5432; G01N 33/531; G01N 33/54353; G01N 33/545; G01N 33/54393

(57) ABSTRACT

The invention provides a novel method of preparing a capture phase for detecting and/or quantifying a target biological entity, said capture phase including a biological ligand for the biological entity, said biological ligand being covalently bonded to an amphiphilic polymer and being immobilized on a solid support, the method being characterized in that the biological ligand is immobilized on the solid support by bringing the solid support into contact with a dispersion of micelles formed by a plurality of chains of the amphiphilic polymer, said micelles carrying a plurality of molecules of the biological ligand on the surface thereof. The invention also provides corresponding capture phases and associated detection methods and kits.

The invention relates to the technical field of detecting biological targets. More precisely, the invention provides a new method of producing capture phases for detecting a biological target in a biological sample, and it also provides the corresponding detection methods and detection kits.

In the field of detection tests, improving the sensitivity and the specificity of detection is an ongoing concern. Such improvements may be obtained by acting on various factors, such as the detection conditions, the capture phase, or the detection phase, and various solutions seeking to solve such problems are proposed in the prior art.

As examples of solutions that have been proposed, reference may be made to the following documents:
Application WO 98/47000 describes a method of revealing a target biological material contained in a sample, in which method a capture phase is provided, said target biological material is brought into contact with at least the capture phase, and the complex comprising the capture phase and the target biological material is detected. Said method is characterized by the fact that the capture phase is in microparticulate or linear form and is constituted by at least a first particulate or linear polymer, having an apparent hydrophilic character and first complexing groups, which groups are bonded via coordination bonding to a first transition metal, which is itself bonded to a first biological ligand capable of specifically recognizing the target biological material. Such a capture phase is proposed in order to optimize fixing of the material thereon, while decreasing or even eliminating any secondary reaction of said material being adsorbed onto said capture phase. In particular, that document proposes using a hydrophilic particulate or linear polymer, and in particular a functionalized polymer obtained by polymerizing a hydrosoluble monomer of acrylamide, of an acrylamide derivative, of methyacrylamide, or of a derivative of methyacrylamide, of at least one cross-linking agent, and of at least one functional monomer. The hydrosoluble monomer is preferably selected from N-isopropylacrylamide, N-ethylmethacrylamide, N-n-propylacrylamide, N-n-propylmethacrylamide, N-isopropylmethacrylamide, N-cyclopropylacrylamide, N,N-diethylacrylamide, N-methyl-N-isopropylacrylamide and N-methyl-N-n-propylacrylamide, the monomer preferably being N-isopropylacrylamide (NIPAM) and the functional monomer preferably being selected from optionally nitrogen-containing carboxylic acids, itaconic acid, acrylic derivatives, and methacrylic derivatives. In preferred manner, use is made of a particulate polymer of the poly(N-isopropylacrylamide) (PNIPAM) type having complexing groups derived from itaconic acid or from maleic-co-methylvinylether anhydride.
Application WO 98/59241 proposes using a capture and/or detection phase having an organic molecule with at least one reactive function and at least one protein material suitable for recognizing or bonding specifically and directly or indirectly with the target biological material, said protein material possessing a specific site for bonding covalently to the reactive function of the organic molecule, which site consists in at least one tag having at least six contiguous lysine residues or lysine derivative residues. Advantageously, the capture phase is immobilized on a solid support by passive adsorption or by covalent bonding. The organic molecule may in particular be a particulate or linear polymer. The following are mentioned as examples of polymers: homopolymers such as polylysine, polytyrosine, copolymers such as maleic anhydride copolymers, N-vinyl-pyrrolidone copolymers, and in particular the copolymer of maleic anhydride and methylvinylether, the copolymer of N-vinyl-pyrrolidone and N-acryloxy succinimide, polysaccharides such as natural or synthetic poly-6-aminoglucose, polynucleotides, and copolymers of amino acids such as enzymes. In the examples, the following polymers are used: AMVE 65: poly(methylvinylether/maleic anhydride); PEAM 86 poly(ethylene/maleic anhydride); SAM 49 poly(styrene/maleic anhydride); and NVPAM 36 poly(N-vinylpyrrolidone/maleic anhydride).
Application WO 03/044533 proposes a method of obtaining a capture phase for a target biological material comprising a modified protein of interest that is capable of specifically bonding, directly or indirectly, with said target biological material and is immobilized on an immobilization phase having reactive groups, in which at least two different peptide sequences, including the peptide sequence of the protein of interest, and one of them including a succession of at least six lysine residues at its N-terminal end and a succession of at least six histidine residues at its C-terminal end, the other one of them including a succession of at least six histidine residues at its N-terminal end and a succession of at least six lysine residues at its C-terminal end, are each immobilized on the immobilization phase by covalent reaction between the primary amine groups of the peptide sequences and the reactive groups of the immobilization phase, and in which the peptide sequence that is coupled to the immobilization phase the most effectively is selected as the capture phase. The immobilization phase corresponds to a polymer, which is then itself immobilized on a solid support. In the examples of that patent application, use is made of a poly(methylvinylether/maleic anhydride) copolymer, the AMVE 67. Since the polymer is not hydrosoluble, it is necessary to dissolve it in anhydrous dimethylsulfoxide (DMSO) prior to the coupling reaction, which is carried out in a 95% aqueous medium with the protein that is the partner of the biological target to be detected.

As described in application WO 01/92361, although those various copolymers serve to improve sensitivity in diagnostic tests, they suffer from a certain number of disadvantages: firstly, the copolymer is adsorbed on the solid support at a plurality of points distributed along the backbone, having the consequence of limiting the availability of biological ligands for reacting with the target biological material. Furthermore, under certain circumstances, the copolymer and biological ligand conjugates exhibit an aggregated structure (see, for example, M. N. Erout et al., Bioconjugate Chemistry, 7 (5), pp. 568-575 (1996) or T. Delair et al., Polymers for Advanced Technologies, 9, pp. 349-361 (1998)). That aggregation phenomenon is completely resolved by the methods implemented in application WO 99/07749, however the sensitivity of the tests for detecting target molecules is affected thereby.

In this context, application WO 01/92361 proposes a novel type of polymer for fixing biological ligands, and presenting:
an architecture that is controlled to keep the biological molecules away from the solid support and enhance the reactivity of the biological ligands with target molecules in solution; and
a size that is sufficient to enable a high degree of grafting of the biological ligands while maintaining a spacing between said ligands, and thus enhancing the sensitivity of diagnostic tests.

Furthermore, in detection techniques such as ELISA, it is essential for the capture phases to conserve the biological activity of the immobilized or coupled biological ligand, after purification and immobilization or coupling, so that it can subsequently interact correctly with the biological target that is to be detected so as to provide detection that is reliable. Specifically when the capture phase includes an immobilized protein, the immobilization of proteins on the capture phase that is carried out at least in part in an organic solvent, as in application WO 01/92361, can lead to denaturing phenomena, ultimately leading to a drop in the sensitivity of the detection method.

In this context, the invention proposes modifying the capture phases used in detection methods, in particular by developing a novel preparation method in order to increase detection sensitivity. For this purpose, a method is proposed in which coupling of the biological ligand to the polymer can be carried out in a solution that is essentially aqueous.

The invention provides a preparation method for preparing a capture phase for detecting and/or quantifying a target biological entity, said capture phase including a biological ligand for the biological entity, said biological ligand being covalently bonded to an amphiphilic polymer and being immobilized on a solid support, the method being characterized in that the biological ligand is immobilized on the solid support by bringing the solid support into contact with a dispersion of micelles formed by a plurality of chains of the amphiphilic polymer, said micelles carrying a plurality of molecules of the biological ligand on the surface thereof.

In the context of the invention, the amphiphilic polymer has a hydrophobic portion oriented towards the core of the micelles and a hydrophilic portion at the surface of the micelles, the biological ligand being covalently coupled to the hydrophilic portion.

Advantageously, in the context of the invention, the immobilization is carried out in a solvent or solvent mixture constituted by at least 90% by weight, preferably at least 95% by weight, and more preferably at least 99% by weight of water. This solvent or solvent mixture corresponds to that present in the dispersion of micelles used for immobilization. Such immobilization in an aqueous phase serves to avoid denaturing the biological ligand, compared with prior art methods that make use of immobilization in a mixture of an aqueous phase and an organic phase, with a high proportion of organic phase.

By way of example, the amphiphilic polymer may be used at a concentration from 50 to 500 μg/mL, while bringing the micelles into contact with the support. This concentration corresponds to the concentration of the dispersion of micelles used.

Advantageously, after immobilization, at least a portion of the polymer remains in the form of micelles, such that micelles formed by a plurality of amphiphilic polymer chains are immobilized at the surface of the support, said micelles carrying a plurality of molecules of the biological ligand on the surface thereof bonded with the amphiphilic polymer in a covalent manner. For this purpose, the concentration of amphiphilic polymer when being brought into contact with the support must be greater than its critical micelle concentration (CMC), so as to maintain the micellar state.

The method of the invention may include a step of covalent coupling between the biological ligand and the amphiphilic polymer, which step is carried out while the polymer is in the form of micelles, so as to form the micelles carrying a plurality of molecules of the biological ligand at the surface thereof. Under such circumstances, in advantageous manner, coupling is carried out in a solvent or solvent mixture constituted by at least 90% by weight, preferably at least 95% by weight, and more preferably at least 99% by weight of water. Once more, such coupling in an aqueous phase serves to avoid denaturing the biological ligand.

Advantageously, coupling is carried out with a concentration of amphiphilic polymer that is at least ten times greater than that used when bringing the micelles into contact with the support, in order to enhance the coupling efficiency, and to guarantee that the micellar state is preserved during coupling (thereby enabling the coupling to be oriented towards the external portion of the hydrophilic ring). Such a concentration serves to enhance conservation of the micellar form during coupling and then during immobilization. By way of example, the amphiphilic polymer may be used at a concentration from 0.5 to 5 mg/mL during the step of covalent coupling with the biological ligand.

The biological ligand is coupled so as to obtain micelles with the hydrophilic portion of the polymer oriented towards the surface of the micelles, and covalently coupled to the biological ligand. Thus, after immobilization, at least a portion of the polymer remains in the form of micelles, such that micelles made up of a plurality of chains of amphiphilic polymer are immobilized at the surface of the support, said micelles carrying a plurality of molecules of the biological ligand on the surface thereof that are covalently bonded to the amphiphilic polymer. In particular, ultimately at least a portion of the micelles are immobilized on the solid support by adsorption by means of an interaction between the biological ligand and the solid support, a portion of the micelles possibly being immobilized on the solid support by adsorption by means of an interaction between the polymer and the solid support, the interactions involved possibly being electrostatic bonds or ionic bonds or hydrophobic interactions, in particular.

Nevertheless, even if a portion of the biological ligands immobilized at the surface of the support are involved in enabling micelles to be immobilized on the solid support, a portion of the biological ligands are free, and in particular not bonded to the support. A fraction of the biological ligands, corresponding in particular to at least 50% of the biological ligands present on the capture phase, remains accessible and becomes available for interacting and bonding with a target biological entity, so as to enable it to be captured when the capture phase is used in a detection method. This is the reason why the support obtained at the end of the method of the invention is referred to as a capture phase.

As an example, in order to enhance immobilization of the polymer in the form of surface micelles, a coupling step as defined above should be carried out with a polymer concentration corresponding to at least 50 times, preferably to at least 200 times the critical micelle concentration of the polymer.

In the context of the invention, the amphiphilic polymer is preferably a linear block polymer including at least one hydrophilic block and at least one hydrophobic block, the hydrophilic block being positioned at the surface of the micelles and carrying at least one molecule of the biological ligand by covalent bonding.

Advantageously, the method is implemented with one or another of the following characteristics, with any combination of the following characteristics, or indeed with all of the following characteristics:
the mean density of biological ligand molecules per polymer chain in the dispersion of micelles is from 0.1 to 100, and in particular from 1 to 100; the mean density of ligand molecules per chain of polymer can be deduced from assaying the residual reactive functions of the ligand involved in coupling;
the micelles in the dispersion and/or the micelles finally immobilized on the support are formed by 100 to 5000 polymer chains and/or carry 10 to 500000 biological ligand molecules;
the dispersion of micelles has a polydispersity index from 0 to 0.2 as determined by dynamic light scattering;
the amphiphilic polymer has a molar mass greater than 5000 g/mol, preferably greater than 10000 g/mol;
the biological ligand is an antigen, a hapten, or a protein;
the amphiphilic polymer includes, or indeed is exclusively constituted by, a first linear block consisting in a hydrophobic homopolymer resulting from polymerizing a hydrophobic monomer A; a second linear block consisting in a hydrophilic copolymer resulting from copolymerizing a monomer B carrying a reactive function X and a hydrophilic monomer C not carrying a reactive function, said second block being bonded to one end of the first block in a covalent manner, in which case, and preferably:
   the monomer A is selected from hydrophobic derivatives of methacrylate, acrylate, acrylamide, methacrylamide, and lactides, or from styrene and its derivatives; and is preferably n-butyl acrylate, tertiobutyl acrylate, tertiobutyl acrylamide, octadecyl acrylamide, lactide, lactide-co-glycolide, or styrene; and/or
   the monomer B is selected from functional derivatives of acrylate, methacrylate, acrylamide or methacrylamide, and from functional styrene derivatives; and is preferably N-acryloxy succinimide, N-methyacryloxy succinimide, 2-hydroxyethyl methacrylate, 2-aminoethyl methacrylate, 2-hydroxyethyl acrylate, 2-aminoethyl acrylate, or 1,2:3,4-di-O-isopropylidene-6-O-acryloyl-D-galactopyranose;
   the monomer B carries a reactive function X selected from —NH₂, —COOH, —OH, —SH, and —CCH functions, ester, halogenocarboynyl, sulfhydryl, disulfide, hydrazine, hydrazone, azide, isocyanate, isothiocyanate, alkoxyamine, aldehyde, epoxy, nitrile, maleimide, halogenoalkyl, and maleimide groups, from functions that can be activated by an activating agent such as carbodiimides, and in particular a carboxylic acid activated in the form of an ester of N-hydroxysuccinimide, pentachlorophenyl, trichlorophenyl, p-nitrophenyl, or carboxyphenyl, or indeed from bifunctional homo- or hetero-compounds;
   the monomer C is selected from hydrophilic derivatives of acrylamide, methacrylamide, N-vinylpyrrolidone, and oxyethylene; the monomer C is preferably N-vinylpyrrolidone or N-acryloyl morpholine;
   the first block has a molar mass between 1000 g/mol and 250000 g/mol;
   the second block has a molar mass greater than 1000 g/mol, and preferably greater than 2000 g/mol; and
   the second block is a random copolymer with a composition, expressed as the ratio of the quantity of monomer C divided by the quantity of monomer B, the quantities being expressed in moles, which ratio is preferably in the range 1 to 10, more preferably in the range 1.5 to 4.

The invention also provides a phase for capturing a target biological entity, the capture phase being characterized in that it comprises micelles immobilized on a solid support, said micelles being formed by a plurality of chains of an amphiphilic polymer, and said micelles carrying a plurality of molecules of at least one biological ligand for the target biological entity on the surface thereof, said molecules of the biological ligand being bonded to the chains of the amphiphilic polymer in a covalent manner.

Advantageously, the capture phase of the invention presents one or another of the following characteristics, any combination of the following characteristics, or indeed all of the following characteristics:
the micelles are immobilized on the solid support by adsorption;
at least a portion of the micelles are immobilized on the solid support by adsorption by means of an interaction between the biological ligand and the solid support, a portion of the micelles optionally being immobilized on the solid support by adsorption by means of an interaction between the polymer and the solid support, the interactions involved possibly being electrostatic or ionic bonds or hydrophobic interactions, in particular; nevertheless, even if a portion of the biological ligands immobilized at the surface of the support are involved in immobilizing the micelles on the solid support, a portion of the biological ligands remain free, and in particular are not bonded to the support; a portion of the biological ligands, corresponding in particular to at least 50% of the biological ligands present on the capture phase, is accessible and available for interacting and bonding with a target biological entity, in order to enable the target biological entity to be captured when the capture phase is used in a detection method; this is in fact why the support on which the micelles are immobilized is referred to as a capture phase;
the micelles immobilized on the support are formed by 100 to 5000 polymer chains and/or carry 10 to 500000 biological ligand molecules;
the amphiphilic polymer has a hydrophobic portion oriented towards the core of the micelles and a hydrophilic portion at the surface of the micelles, the biological ligand being coupled in a covalent manner to the hydrophilic portion;
the amphiphilic polymer is a linear block polymer including at least one hydrophilic block and at least one hydrophobic block, the hydrophilic block being positioned on the surface of the micelles, and carrying at least one molecule of the biological ligand by covalent bonding;
the amphiphilic polymer has a molar mass greater than 5000 g/mol, preferably greater than 10000 g/mol;
the amphiphilic polymer includes, or indeed is constituted exclusively by, a first linear block consisting in a hydrophobic polymer resulting from polymerizing a hydrophobic monomer A; and a second linear block consisting in a hydrophilic copolymer resulting from copolymerizing a monomer B carrying a reactive function X with a hydrophilic monomer C not carrying a reactive function, said second block being bonded to one end of the first block in a covalent manner; the monomers A, B, C and the reactive functions X, and the blocks are preferably as defined above in the context of the method; and
the biological ligand is an antigen, a hapten, or a protein.

The invention also provides a device for detecting and/or quantifying a target biological entity, the device comprising a capture phase of the invention and at least one tracer for detection.

The invention also provides a device for detecting and/or quantifying a target biological entity, comprising a capture phase obtained by the method of the invention and at least one tracer for detection.

The invention also provides a kit for detecting and/or quantifying a target biological entity, the kit comprising:
a solid support;
a dispersion in aqueous solution of micelles formed by chains of an amphiphilic polymer, carrying a plurality of molecules of at least one biological ligand for the target biological entity on the surface thereof, said biological ligand molecules being bonded to the chains of the amphiphilic polymer in a covalent manner; and
at least one tracer for detection.

The dispersion and the solid support preferably present the same characteristics as those specified in the present description with reference to the method of preparing the capture phase.

The invention also provides a method of detecting and/or quantifying a target biological entity in vitro in a biological sample, wherein: a capture phase of the invention is provided; said biological sample is brought into contact with at least the capture phase; and said target biological entity fixed on the capture phase is detected and/or quantified after the biological entity has bonded with a biological ligand molecule covalently bonded to the chains of the amphiphilic polymer of the capture phase.

The invention also provides a method of detecting and/or quantifying a target biological entity in vitro in a biological sample, wherein: a capture phase obtained by the method of the invention is provided; said biological sample is brought into contact with at least the capture phase as obtained in this manner; and said target biological entity fixed on the capture phase is detected and/or quantified after the biological entity has bonded with a biological ligand molecule covalently bonded to the chains of the amphiphilic polymer of the capture phase.

The invention also provides a detection method for detecting and/or quantifying a target biological entity in vitro in a biological sample, wherein: a capture phase is prepared by the method of the invention; said biological sample is brought into contact with at least the capture phase as prepared in this way; and said target biological entity fixed on the capture phase is detected and/or quantified after the biological entity has bonded with a biological ligand molecule covalently bonded to the chains of the amphiphilic polymer of the capture phase.

Such detection and/or quantification methods may be a direct method in which the sample that might contain the target biological entity is brought into contact with the capture phase and bonding between the biological ligand immobilized on the support and the target biological entity is revealed by the presence of a tracer. Under such circumstances, the tracer is in particular a biological ligand of the target biological entity coupled to a marker. Sandwich immunoassays, also known as immunometric assays, are the formats that are the most conventionally used.

Such detection and/or quantification methods may be an indirect method in which the sample that might contain the target biological entity is brought into contact with the capture phase in the presence of an analog of the target biological entity, and bonding between the biological ligand immobilized on the support of the capture phase and the target biological entity is revealed by the presence of a tracer, indirectly by detecting the bonding between the biological ligand immobilized on the support and the analog of the target biological entity. Under such circumstances, the tracer is in particular the analog of the target biological entity coupled to a marker. Competitive immunoassays are the formats that are the most conventionally used.

Whether in a direct method or an indirect method, the marker in the tracers employed is selected, by way of example, from enzymes, chromophores, radioactive molecules, fluorescent molecules, and electrochemiluminescent salts.

An improvement in detection sensitivity has been observed using the capture phase described or obtained in the context of the invention. This improvement in sensitivity can be explained in various ways: because the biological ligand is not denatured, it can be coupled and immobilized in an aqueous medium because the biological ligand is presented better, thereby enhancing its subsequent interaction and bonding with the target biological entity, or because of these various effects in combination.

The invention can be better understood from the following more detailed definitions and descriptions.

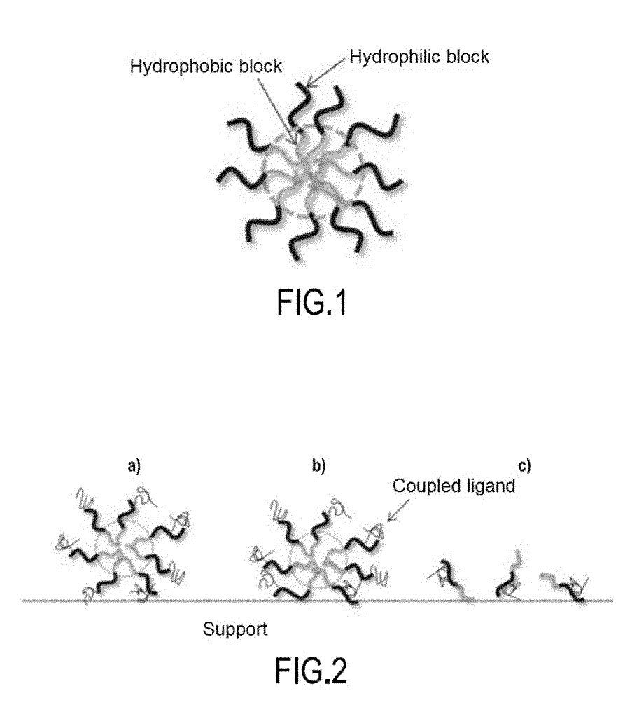

The term “micellar formed by a plurality of chains of an amphiphilic polymer” is used to mean a spheroidally-shaped assembly of chains of an amphiphilic polymer, in which a hydrophilic portion forms the crown (directed towards the aqueous solution in which the micelles are to be found), and a hydrophobic portion forms the core, as shown diagrammatically in FIG. 1. An amphiphilic polymer self-assembles into this shape provided that it is in an aqueous solution at a concentration that is greater than a characteristic concentration for said polymer, known as the critical micelle concentration. In the context of the invention, micelles are characterized by their hydrodynamic diameter. This is calculated on the basis of the hydrodynamic radius, which is measured by the dynamic light scattering technique. The hydrodynamic radius is the radius of a theoretical sphere having the same diffusion coefficient as the particle under consideration. In the context of the invention, the micelles generally present a hydrodynamic diameter from to 200 nm, preferably from 50 to 150 nm. The hydrodynamic diameter of the micelles can be measured in a dispersion of 50 μg/mL of polymer in a 1 mM aqueous solution of NaCl, e.g. using a ZetasizerNano© S90 instrument (Malvern, UK) at a temperature of 25° C. The polydispersity index (defined as the square of the ratio of the standard deviation divided by the hydrodynamic diameter as determined by the same instrument), which is representative of the width of the size distribution, is typically less than 0.2, and is more often less than 0.1, which is characteristic of a narrow size distribution for such micelles.

The term “amphiphilic polymer” is used to mean a polymer that possesses both a hydrophilic portion or block and a hydrophobic portion or block. The amphiphilic character of the polymer, in an aqueous solution and above a concentration referred to as the critical micelle concentration (CMC), is characterized by its aggregation in the form of micelles, which process serves to reduce the free energy of the system. In the context of the invention, the amphiphilic polymer chains are preferably linear and not branched so as to enhance the way they assemble together in the form of micelles. An amphiphilic polymer suitable for use in the context of the invention is a copolymer constituted by at least two linear blocks, at least one generally hydrophobic block and at least one generally hydrophilic block. In a linear block, each monomer, with the exception of the end monomers, is bonded to two other monomers, said monomer lying between the two other monomers along the chain. One of the ends of the generally hydrophobic block is covalently bonded to one of the ends of the generally hydrophilic block.

Unless specified otherwise, the term “copolymer”, should be understood to mean a polymer made up of at least two different monomers. The term “copolymer” thus equally encompasses random copolymers, block copolymers, or alternating polymers.

Such an amphiphilic polymer may include a first hydrophobic block in the form of a hydrophobic homopolymer, i.e. comprising a chain of a single hydrophobic monomer A.

Such an amphiphilic polymer may include a second block providing the hydrophilic component of the polymer, in the form of a copolymer constituted by two monomers: a first monomer C supplying the hydrophilic character, in order to enhance maximum deployment of the second block in an aqueous phase, and another monomer B that provides a reactive function X in order to provide covalent coupling with the biological ligand. This second block is preferably a random copolymer, and advantageously it has reactive functions close to the end of the hydrophilic block that are not bonded to the hydrophobic block (i.e., in the micelle, the end that is deployed towards the solution), the number of said reactive functions being sufficient to ensure that the presentation and/or accessibility of the ligand after coupling in the solution is suitable for obtaining increased recognition efficiency. In an extreme situation, the second block may thus be constituted by a single non-functional monomer, with only the monomer situated at the end of the chain (on the end that is not bonded to the hydrophobic block) being functionalized. By way of example, this applies to a hydrophilic block of polyethylene glycol PEG (non-functional monomer unit —CH₂—CH₂—O—) in which the end of the hydroxyl chain can be used for conjugation with ligands.

Advantageously, at least one reactive function X, and preferably 1 to 100 reactive functions X are present on each amphiphilic polymer chain.

Preferably, the second block of the amphiphilic polymer is a random copolymer (in which the monomer motifs B and C are distributed randomly along the macromolecular chain), or an alternating copolymer (in which the monomers B and C follow one another regularly with a general structure (BC)n, where n is an integer).

The various copolymers may be obtained by means of a polycondensation reaction, or by chain polymerization using a radical technique, an ionic technique, or by group transfer, advantageously by living radical polymerization such as reversible termination polymerization (nitroxide-mediated polymerization, NMP), atom transfer radical polymerization (ATRP), and preferably by reversible addition-fragmentation chain-transfer polymerization, called RAFT (see WO 98/01478). By way of example, these various polymerization techniques are described in K. Matyjazewski, Controlled Radical Polymerization, American Chemical Society Series, Washington D.C., USA, 1997; and by G. Odian, Principles of Polymerization, Third edition, Wiley-Interscience Publication, 1991. Application WO 01/92361 describes the preparation of such amphiphilic polymers, and reference may be made thereto for further details.

The term “hydrophobic monomer” is used to mean a monomer for which the homopolymer, when in an aqueous phase, presents a compact ball structure corresponding to a Mark-Houwink-Sakurada coefficient (form factor) of less than 0.8.

The term “functional monomer” is used to mean a monomer carrying a reactive function X.

The term “hydrophilic monomer” is used to mean a monomer in which the homopolymer presents, in an aqueous phase, a structure that is deployed, corresponding to a Mark-Houwink-Sakurada coefficient greater than 0.8.

The technique used for measuring the molar mass of a polymer, expressed in the present invention as Mn (number average molar mass in g/mol) is steric exclusion chromatography (SEC) with a refractometric detector, giving molar masses that are quoted relative to a calibration (e.g. a polystyrene standard in an organic phase).

The term “biological ligand” is used to mean a biological entity capable of recognizing or bonding with the target biological entity. The biological ligand is thus a bonding partner for the target biological entity. As examples of such biological ligands, mention may be made in particular of a protein or glycoprotein material such as an antigen, a hapten, an antibody, a protein, a nanofitin, a peptide, an enzyme, a sugar and fragments thereof, and a lectin; and also a nucleic material such as a nucleic acid (DNA or RNA), a fragment of nucleic acid, a probe, or a primer. The invention is particularly adapted to biological ligands selected from antigens, haptens, and proteins.

The term “target biological entity” is used to mean a biological material of interest. As examples of such biological materials of interest, mention may be made of antibodies, receptors, haptens, antigens, proteins, peptides, enzymes, sugars, and nucleic acids.

The antibodies acting as a biological ligand for the target biological entity are polyclonal antibodies or monoclonal antibodies.

Polyclonal antibodies may be obtained by immunizing an animal using, as the immunogen, the target biological entity, a fragment thereof, or indeed an equivalent that is close in structural terms, followed by recovering the antibodies under investigation in purified form by taking serum from said animal and separating said antibodies from the other components of the serum, in particular by column affinity chromatography, on which an antigen specifically recognized by the antibodies is fixed, in particular the immunogen.

Monoclonal antibodies may be obtained by the hybridoma technique, which is well known to the person skilled in the art. The monoclonal antibodies may also be recombinant antibodies obtained by genetic engineering, using techniques that are well known to the person skilled in the art.

As examples of antibody fragments, mention may be made of the fragments Fab, Fab′, F(ab′)2, and also of single-chain variable fragments (scFv) and double-stranded variable fragments (dsFv). These functional fragments may in particular be obtained by genetic engineering.

Nanofitins (trade name) are small proteins that, like antibodies, are capable of bonding to a biological target, thus making it possible to detect it, to capture it, or merely to target it within an organism.

The term “hapten” designates non-immunogenic compounds, i.e. compounds that are not themselves capable of promoting an immune reaction by producing antibodies, but that are capable of being recognized by antibodies obtained by immunizing animals under known conditions, in particular by immunization with a hapten-protein conjugate. These compounds generally have a molecular mass of less than 3000 Da, and more usually less than 2000 Da, and by way of example they may be glycosylated peptides, metabolites, vitamins, hormones, prostaglandins, toxins or various medications, nucleosides, and nucleotides.

The term “lectin” is used to mean proteins that bond specifically and reversibly to certain sugars. They play a major role in immunity, by recognizing the specific carbohydrates of certain pathogenic infectious agents. An example of a lectin is concanavalin A (hemagglutinin), which is responsible for hemagglutination, amongst other things.

The biological ligands used may optionally be specific to the target biological entity. They are said to be specific when they are capable of bonding exclusively or quasi-exclusively with the target biological entity. They are said to be non-specific when the selectivity of the bonding with the target biological entity is weak, and they are then capable of bonding with other biological entities such as other proteins or antibodies. In general manner, it is preferred to use a biological ligand that is specific for the target biological entity.

Naturally, the biological ligand that behaves as a bonding partner for the target biological entity is selected as a function of the target biological entity that it is desired to detect:
if the target biological entity is an antibody, then the biological ligand is an antigen that recognizes said antibody, preferably specifically;
if the target biological entity is an antigen, then the biological ligand is an antibody that recognizes said antigen, preferably specifically;
if the target biological entity is a receptor, then the biological ligand is a protein that bonds with said receiver, preferably specifically; and
if the target biological entity is a hapten, then the biological ligand is an antibody or a protein that recognizes said hapten, preferably specifically.

The invention is particularly suitable for situations in which the biological ligand is an antigen, a hapten, or a protein.

The term “reactive function” as present firstly on the polymer and secondly on the biological ligand, is used to mean either a function that is capable of forming a covalent bond by reacting with another reactive function, or else the functions that can be activated and that lead to a reactive function after being activated. By way of example, such reactive functions X may be selected from the following groups: ester, halogenocarbonyl, sulfhydryl, disulfide, amine (NH₂), carboxylic acid (COOH), hydrazine, hydrazone, azide, isocyanate, isothiocyanate, alkoxyamine, aldehyde, epoxy, nitrile, maleimide, halogenoalkyl, hydroxyl, thiol, alkyne (—C≡CH—), maleimide, and functions that can be activated by an activating agent such as carbodiimides or homo- or hetero-bifunctional compounds. It is possible in particular to use an activated carboxylic acid in the form of an ester of a N-hydroxysuccinimide ester, a pentachlorophenyl ester, of trichlorohenyl ester, a p-nitrophenyl ester, or a carboxyphenyl ester.

The reactive function(s) present on the biological ligand and enabling a covalent bond to be formed between the polymer of the micelles and the biological ligand may exist naturally on the biological ligand. For example, for a biological ligand of the protein type with a sufficient lyzine composition, the amines carried by the side chain of the lysine may be used for coupling. Nevertheless, in numerous situations, the reactive functions need to be “introduced” beforehand, e.g. in the form of a tag, using techniques well known to the person skilled in the art. A tag may be defined as an added sequence of amino acids, i.e. a sequence added to the original structure of a protein used as a biological ligand, and introduced into a special location of said original structure in order to enable it to be exposed in a manner that is pertinent, specifically for covalent fixing on the polymer. When the biological ligand is a protein material, it is possible, for example, to use a tag having six or more lyzine residues, or lyzine derivative residues, and possibly other amino acids. Such tags may be found at any location on the protein. When the biological ligand is a protein, the tag is preferably situated at its N-terminal or C-terminal end.

Numerous methods are available for introducing reactive functions to a biological ligand: for proteins, antigens, antibodies, or polypetides, see, for example, “Chemistry of Protein Conjugation and Cross-linking” by S. S. Wong, CRC Press, Boca Raton, 1991, or “Bioconjugate Techniques”, by G. T. Hermanson, Academic Press, San Diego, 1996. For nucleic acids, it is possible, for example, to synthesize a polynucleotide by a chemical method on a solid support having a reactive function at any location on the chain, such as, for example, the 5′ end or the 3′ end, or on a base, or on an internucleotide phosphate, or on the 2′ position of the sugar (see “Protocols for Oligonucleotides and Analogs, Synthesis and Properties” edited by S. Agrawal, Humana Press, Totowa, N.J.). Methods of introducing reactive functions onto haptens are given in particular in “Preparation of Antigenic Steroid-protein Conjugate”, by F. Kohen et al., in Steroid Immunoassay”, Proceedings of the Fifth Tenovus Workshop, Cardiff, April 1974, edited by E. H. D. Cameron, S. H. Hillier, K. Griffiths, such as, for example, introducing a hemisuccinate function in position 6, 11, 20, or 21, a chloroformate in position 11, or a carboxymethyl function in position 6, for progesterone.

The reactive function that is present on the polymer, and the reactive function that is present on the biological ligand, are selected so as to react with each other and form a covalent bond establishing a permanent bond between the polymer chains of a micelle and the biological ligands. As an example, a primary amine function may be coupled to an activated carboxylic acid, in particular by N-hydroxy succinimide, or to an aldehyde; an alkoxyamine function may be coupled with a ketone or with an aldehyde; a hydrazine function may be coupled with an aldehyde; or indeed a thiol function may be coupled to a halogenoalkyl or a maleimide. In known manner, when coupling between an amine and an aldehyde, it is preferable to reduce the imine that is formed, either simultaneously by the action of NaBH₃CN, or else in a later step by the action of NaBH₄ or NaBH₃CN.

Advantageously, on average, 0.1 to 100 molecules of biological ligand are fixed per polymer chain, and on average the micelles carry 10 to 500000 molecules of biological ligand depending on their molar mass and on their nature. By way of example, on the micelles comprising the polyactide-b-poly(N-vinylpyrrolidone-co-N-acryloxy succinimide) copolymer (PLA-b-P(NVP-co-NAS)) that is used in the examples given below (having a chain that includes on average 80 reactive functions of the N-hydroxy succinimide ester type), it has been possible to couple (in PBS pH 7.4) about 7500 p24 proteins per micelle (molar mass of p24: 24000 g/mol), i.e. about one protein per chain. This number of ligands can be determined by assaying the residual amine functions of the ligand after the coupling reaction (2,4,6-trinitrobenzenesulfonic acid or fluorescamine assaying method) and can be associated with an electrophoresis gel SDS-PAGE when coupling proteins. Naturally, given that the polymer chains usually carry a plurality of reactive functions, a plurality of biological ligand molecules may become fixed on a polymer chain, thereby multiplying the number of biological ligand molecules that are fixed per micelle.

Since the coupling takes place on micelles of copolymer, attaching the ligand near to the end of the hydrophilic block, which is spread out in solution, is enhanced, thereby facilitating future recognition by the target. Coupling of the ligand is correspondingly more effective with increasing number of reactive functions on the hydrophilic block (in particular on its outer portion that is the most deployed in solution). The micellar state of the polymer during coupling of the ligand serves to orient fixing of the ligand towards the end of the hydrophilic chain of the polymer, thereby enhancing its accessibility in solution.

In order to control both the size and the size distribution of the polymer chains, and thus control the size distribution of the micelles that are obtained from the polymer chains, it is preferable to use controlled polymerization techniques for preparing the polymers, such as nitroxide-mediated polymerization (NMP), atom transfer radical polymerization (ATRP), radical polymerization by reversible addition-fragmentation chain-transfer (RAFT), or indeed ionic polymerization.

In most circumstances, the copolymer is obtained in two stages. In the examples below, the hydrophobic first block, poly(D,L-lactide), is obtained by ring opening polymerization of a D,L-lactide monomer that is functionalized at the end of the chain by an SG1 nitroxide fragment that is capable of initiating NMP copolymerization of the pair of monomers of N-vinyl-pyrrolidone (NVP, hydrophilic monomer) and of N-acryloxy succinimide (NAS, functional monomer, carrying N-hydroxy succinimide ester functions).

Micelles may be formed from amphiphilic polymer using any known technique. In particular, the common solvent method that is the most used is described below (G. Riess, Prog. Polym. Sci. 28, pp. 1107-1170, (2003)). The polymer is dissolved in a solvent, usually an organic solvent, serving to dissolve both the hydrophobic portion and the hydrophilic portion of the polymer, so as to dissolve the polymer. Such a solvent may be selected from water-miscible solvents (acetone, acetonitrile, 1,4-dioxane, tetrahydrofuran (THF), or indeed dimethylsulfoxide (DMSO)) and it should be selected as a function of the nature of the amphiphilic copolymer. Water is then added, leading to micelles being formed, and then the initial solvent is eliminated by evaporation under reduced pressure (for solvent(s) of sufficient volatility, of the acetone, acetonitrile, THF type) or by dialysis against water (for “heavier” solvent(s) of the DMSO type). Typically, the concentration range for the copolymer in the organic solvent is 1 mg/mL to 15 mg/mL, and the volume ratios of organic solvent to water are from 1 to 0.2. Such a technique is described for a polymer of the polyactide-b-poly(N-vinyl pyrrolidone-co-N-acryloxy succinimide) type (PLA-b-P(NAS-co-NVP)), in the publication by N. Handké et al. Macromol. Biosci., 13, pp. 1213-1220 (2013), to which reference may be made for further details. Other well-known methods of preparing micelles could be used, and in particular the dialysis method, which consists in placing the copolymer in solution in a common solvent in a dialysis device, and carrying out dialysis against water. The method of direct dissolution of the copolymer in water could also be used for copolymers having a high hydrophilic/hydrophobic balance.

In known manner, the solid support may be in any appropriate form such as a plate, a cone, a bead, the bead possibly being radioactive, fluorescent, magnetic, and/or conductive, a bar, a glass tube, a well, a sheet, a chip, a microtitration plate, or the like. When the support is in the form of beads, they usually have a diameter from about one hundred micrometers to one nanometer. The material of the support is preferably selected from latexes, polystyrenes, styrene/butadiene copolymers, styrene/butadiene copolymers mixed with one or more polystyrenes, polypropylenes, polycarbonates, polystyrene/acrylonitrile copolymers, styrene/methyl methacrylate copolymers; from synthetic and natural fibers; from polysaccharides and cellulose derivatives; from glass, silicon, and derivatives thereof.

The biological micelle-ligand conjugate is immobilized on the solid support using any appropriate means, usually by adsorption. This type of immobilization may be carried out by “passive” adsorption onto the solid phase, in particular by means of interactions of the hydrophobic, electrostatic, or Van der Waals type or by hydrogen bonding, the relative contribution of which depends on the nature of the amphiphilic polymer, the coupled ligand, and the solid immobilization support. The same conditions as those applied and routinely used by the person skilled in the art for immobilizing a biological ligand on a solid support may be carried out. In particular, it is possible to deposit a dispersion of micelles carrying biological ligands in a buffered aqueous solution. Advantageously, the buffered aqueous solution is formed by a solvent or a mixture of solvents constituted by at least 90% by weight, preferably at least 95% by weight, and more preferably at least 99% by weight water. Buffers that are in routine use in the field of diagnostics may be used, in order to obtain and stabilize the pH in the desired range as a function of the nature of the biological ligand. As an example, a PBS (phosphate buffer saline) or tris (tris-hydroxymethylaminomethane) buffer could be used. At the concentrations of copolymer employed, greater than the CMC of the polymer, it is reasonable to assume that adsorption is essentially regulated by the interaction between the surface ligand of the micelles and the solid phase. These interactions (of the electrostatic and/or hydrophobic type, etc.) are primarily the same as those that are in play for the free ligand, deposited alone onto the solid capture support. The micellar state is most generally conserved on the support, as shown in the majority of studies viewing micelles by electron microscopy and/or atomic force microscopy, which require deposition on a support (Cho et al, J Am Chem Soc, 128, pp. 9935-9942 (2006), Gensel et al, Soft Matter, 7, 11144 (2011)). However, depositing a fraction of a copolymer-ligand conjugate deposited in the form of a unimer should not be excluded, particularly as a function of the nature of the copolymers, ligands and supports used (Freij-Larsson, Biomaterials, 17, pp. 2199-2207 (1996)). Nevertheless, immobilization leads to micelles formed from amphiphilic polymer carrying on their surface a plurality of molecules of biological ligand immobilized on the solid support even if a portion of the amphiphilic polymer has lost its micellar organization.

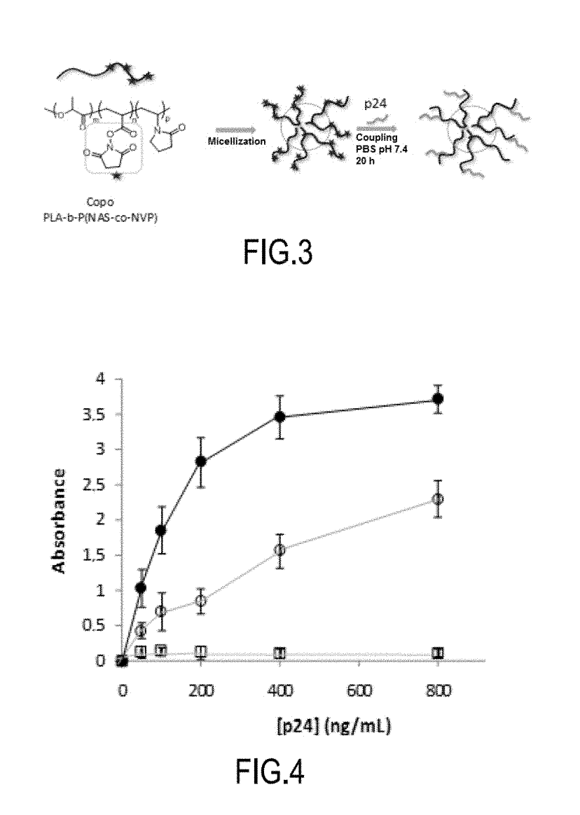

Ultimately, at least a portion of the micelles are still formed and are immobilized on the support, principally via support-ligand interactions, and to a lesser extent potentially by support-polymer interactions, as illustrated in FIG. 2.

The capture phases described or obtained in the context of the invention could be used with the aim of detecting and/or assaying and/or purifying a target biological entity. The user could directly provide a solid onto which the micelle-biological ligands conjugate is immobilized, or provide a kit comprising a solid support and a dispersion of the micelle-biological ligands conjugates described in the context of the invention in an aqueous solution, and then carry out the immobilization personally. The aqueous solution is advantageously constituted by a solvent or mixture of solvents constituted by at least 90% by weight, preferably at least 95% by weight, and more preferably at least 99% by weight of water. Here again, this is a buffered aqueous solution as described above.

The capture phases described or obtained in the context of the invention could be used in any technique for detection and/or quantification of a target biological entity in a biological sample. The term “quantification” that the concentration of the biological entity that is present is determined. The term “biological sample” means any animal biological sample, preferably human, that is susceptible of containing a biological entity of interest. These samples are well known to the person skilled in the art. They may correspond to a sample of a biological fluid, for example whole blood, serum, plasma, urine, cerebrospinal fluid, an organic secretion, a tissue sample, or isolated cells. This sample may be used as is, or else, prior to carrying out the detection method and/or quantification method, it may undergo an enrichment or culture type preparation, using methods known to the person skilled in the art. The samples employed in the detection and/or quantification methods may optionally, in fact, have already been modified before they are used. Examples of samples that have not previously been modified and that may be mentioned are biological fluids such as whole blood, and examples of already modified samples that may be mentioned are serum, plasma, cells that are recovered from a biopsy, or following surgery, and that are cultured in vitro. The detection or quantification method could then be carried out in the culture supernatant or indeed in the cell lysate.

In addition to the capture phase, the detection and/or quantification techniques in general use a tracer or detection phase in order to detect immobilization of the target biological entity on the capture phase. Detection phases or tracers of this type comprise a marker.

The detection and/or quantification method could employ direct or indirect detection.

In a direct detection method, the sample that is suspected of containing the target biological entity is brought into contact with the capture phase and the bond between the biological ligand immobilized on the support and the target biological entity is then revealed by the presence of a tracer. The tracer generally corresponds to a biological ligand of the target biological entity (usually different from the biological ligand immobilized on the support at the capture phase) coupled to a marker.

Indirect detection methods, also known as competitive methods, are also assays that are very familiar to the person skilled in the art, and in particular are used when the target biological entity is a hapten. It consists in assaying the target biological entity in the sample by generating a competition between the target biological entity and the sample and an analog of that target biological entity. Under such circumstances, the sample that is suspected of containing the target biological entity is brought into contact with the capture phase in the presence of an analog of the target biological entity. The bond between the biological ligand immobilized on the support and the target biological entity is then revealed because of the presence of a tracer, indirectly by detecting the bond between the biological ligand immobilized on the support and the analog of the target biological entity.

The analog of the target biological entity is used in the competition reaction after coupling with a marker in order to form a conjugate or tracer. The measured signal emitted by the tracer is then inversely proportional to the quantity of target biological entity of the sample.

The term “marker” means any molecule that is capable, directly or indirectly, of generating a detectable signal. A non-limiting list of these direct detection markers is as follows:
enzymes that produce a detectable signal, for example by colorimetry, fluorescence, or luminescence, such as horseradish peroxidase, alkaline phosphatase, β-galactosidase, or glucose-6-phosphate dehydrogenase;
chromophores such as fluorescent, luminescent, or colorant compounds;
radioactive molecules such as ³²P, ³⁵S or ¹²⁵I;
fluorescent molecules such as Alexa dyes or phycocyanins; and
electrochemiluminescent salts such as organometallic derivatives based on acridinium or ruthenium.

Indirect detection systems may also be used such as, for example, ligands that are capable of reacting with an anti-ligand. The ligand then corresponds to a marker in order to constitute the tracer along with the analog of the target biological entity.

Ligand/anti-ligand pairs are well known to the person skilled in the art as is the case, for example, with the following pairs: biotin/streptavidin, hapten/antibody, antigen/antibody, peptide/antibody, sugar/lectin, polynucleotide/polynucleotide complement.

The anti-ligand may then be detectable directly by the direct detection markers described above, or may itself be detectable by another ligand/anti-ligand pair, and so on.

Under certain conditions, these indirect detection systems may lead to amplification of the signal. Signal amplification techniques of this type are well known to the person skilled in the art; in this regard, reference may be made to the Applicant's prior patent applications FR 2 781 802 or WO 95/08000.

Depending on the type of marking used, the person skilled in the art should add reagents enabling the marking to be visualized or the emission of a signal detectable by any appropriate type of measuring instrument such as, for example, a spectrophotometer, a spectrofluorimeter or indeed a high definition camera.

In conventional manner, in order to determine the quantity of target biological entity present in a sample, the signal, which is proportional (in a direct process) or inversely proportional (in an indirect process) to the quantity of target biological entity of the sample, may be compared with a calibration curve that has already been obtained using techniques that are well known to the person skilled in the art. Thus, for example, the calibration curve is obtained by carrying out an assay using the same biological ligand, as well as known increasing quantities of the target biological entity. A curve is then obtained by placing the concentration of target biological entity along the abscissa, and the corresponding signal obtained after assay up the ordinate.

The detection/quantification process in accordance with the invention may be applied directly to the format for commercial tests that are available for detection/quantification of the target biological entity. Particular examples of a biological ligand to which the invention may be applied that may be mentioned are the proteins p24, and gp120 from HIV, the core proteins NS3, NS4 and NS5 of the hepatitis C virus, the proteins ORF2 and ORF3 of the hepatitis E virus, and the oligosaccharide galactomannan from fungi of the genus Aspergillus. The target biological entities are then immunoglobulins (humoral response) directed against these pathogens. Other particular examples of a biological ligand to which the invention may be applied are biomarkers of human pathologies such as the protein S100B, the troponins I and T, anti-Mullerian hormone (AMH), procalcitonin, PSA (prostate-specific antigen) and other tumor markers. Under these circumstances, the target biological entities are auto-antibodies. Clearly, it is possible to apply the invention to related biomarkers in animals.

The following examples, made with reference to the accompanying figures, illustrate the invention and demonstrate its importance in obtaining improved sensitivity of detection.

FIG. 1 is a diagrammatic representation of micelles carrying biological ligands, in a block type amphiphilic copolymer.

FIG. 2 is a diagrammatic representation of micelles carrying biological ligands immobilized on the surface of a support, which highlights the interactions between the micelles and the support: A) via the ligand present on the surface of the micelles; B) via an amphiphilic polymer (via a hydrophilic chain); C) by adsorption in the form of a unimer (via a hydrophobic/hydrophilic/or ligand portion).

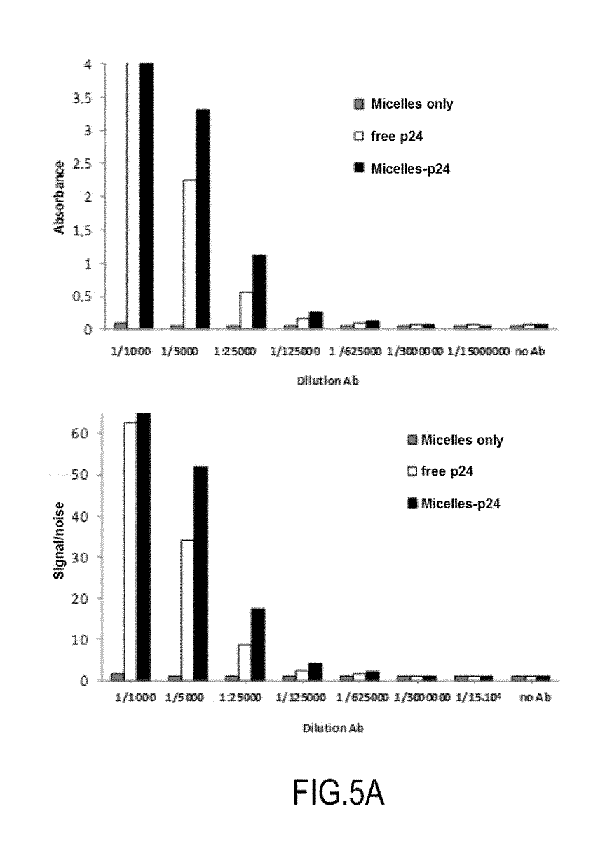

FIG. 3 illustrates the preparation of micelles coupled with p24, used in the examples below.

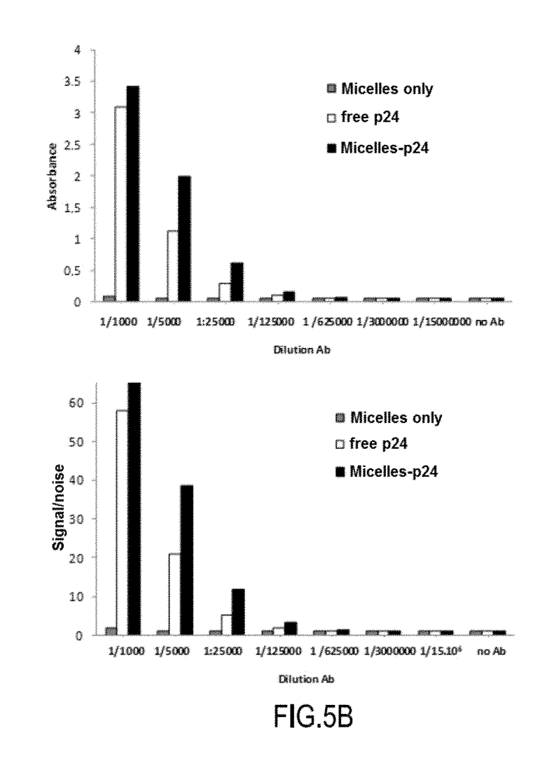

FIG. 4 presents the results obtained with an ELISA test with free p24 (o) or p24 coupled with captured micelles (•) as well as with micelles alone (□) (under the same dilution conditions as with p24).

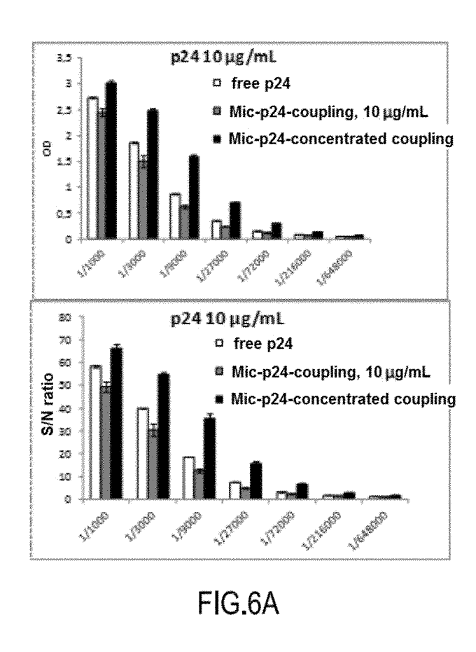

FIGS. 5A and 5B present the results obtained with the same format for the ELISA test as in FIG. 4, but varying the concentration of detection antibody, with immobilization carried out with a concentration of p24 of a):10 μg/mL (FIG. 5A), b): 1 μg/mL (FIG. 5B); graphs of the pure absorbance signal and signal-to-noise ratio as a function of the dilution of antibody (Ab).

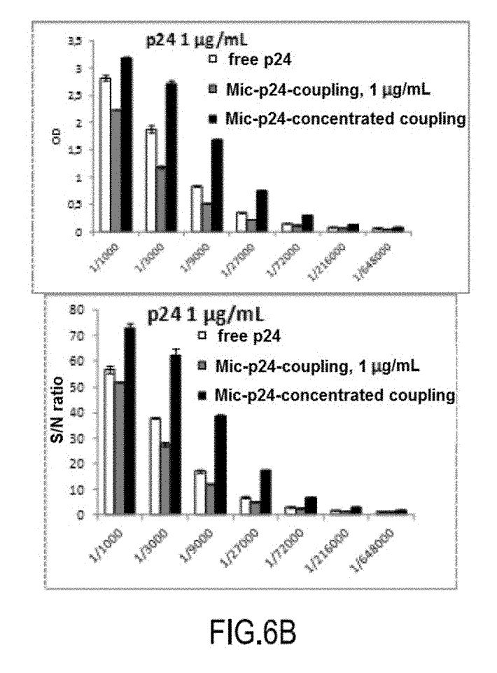

FIGS. 6A and 6B present the influence of the coupling conditions (dilute or concentrated) of p24 on the increase in sensitivity using ELISA after immobilization carried out with a concentration of p24 of: a):10 μg/ml (FIG. 6A), b): 1 μg/ml (FIG. 6B); graphs of the pure absorbance signal and signal-to-noise ratio as a function of the dilution of antibody.

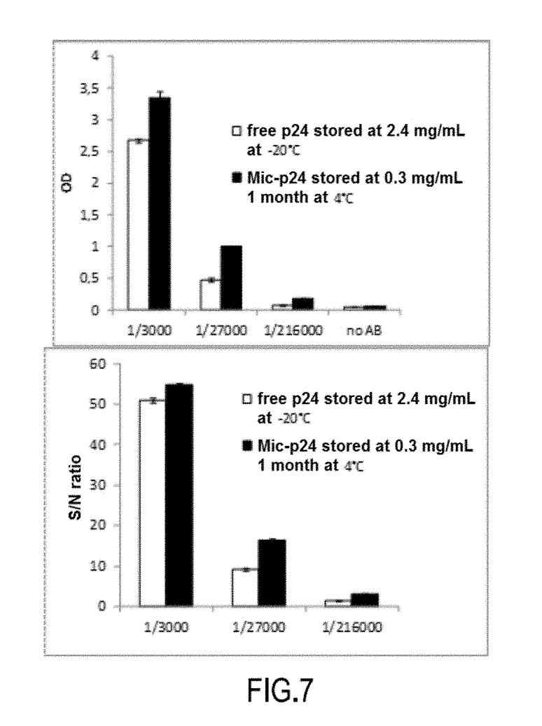

FIG. 7 demonstrates the stability of micelles-p24 evaluated using ELISA after immobilization carried out with a concentration of p24 of 1 μg/ml; graphs of the pure absorbance signal and signal-to-noise ratio as a function of the dilution of antibody.

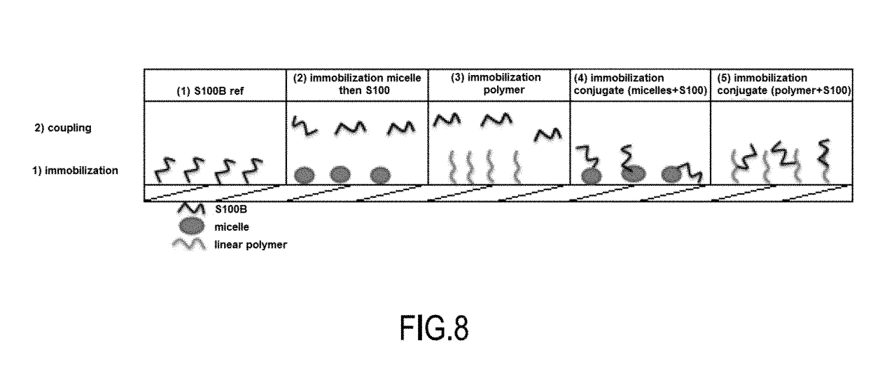

FIG. 8 presents various conditions for immobilization of the antigen S100B on the solid support used below.

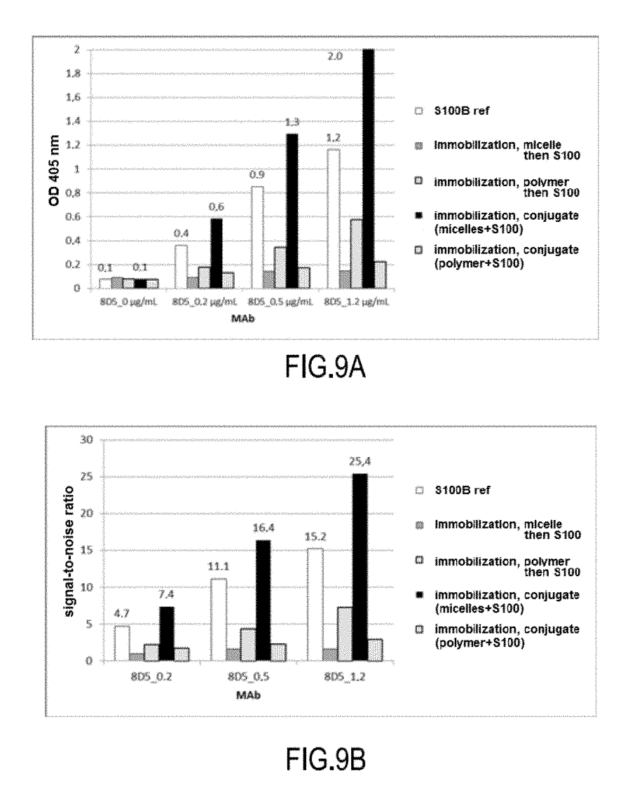

FIGS. 9A and 9B present the ELISA results for the various types of immobilization carried out in accordance with FIG. 8; graphs of the pure absorbance signal and signal-to-noise ratio as a function of the concentration of antibody (Mab).

### EXAMPLES

A study pertaining to the use of micelles formed from polylactide-b-poly(N-vinylpyrrolidone-co-N-acryloxysuccinimide) copolymer (PLA-b-P(NAS-co-NVP)) (with respective molar masses of 19000 and 22000 g·mol⁻¹ for the PLA and P(NAS-co-NVP)) blocks was carried out. The micelles were prepared using the common solvent method (acetonitrile). Coupling of the proteins, acting as the biological ligand for the target antibody, was carried out via their lysine or N-terminal amine functions (see FIG. 3) with the reactive N-succinimidyl (NS) ester functions of the NAS units present on the crown of the micelles.

The effect of using micelle-protein conjugates on the increase in the sensitivity of the immuno-enzymatic tests in the capture phase was demonstrated:

1. for 3 different sources of antigens:

a. recombinant p24, capsid protein of HIV;

b. troponin I cardiac complex I-T-C from Hytest;

c. native protein S100B bovine brain extract, from HyTest;

2. On 2 immunoassay techniques:

a. manual ELISA technique;

b. automated VIDAS technique marketed by bioMérieux.

### ELISA Study on the Detection of Anti-p24 Antibody

Coupling of P24 Antigen to Copolymer Micelles

### Preparation of Micelles

The micelles were prepared using the common solvent method (or nanoprecipitation). The copolymer (20 mg) was dissolved in 2 mL of acetonitrile, then this solution was added to 4 mL of milli-Q water at a regular rate. The acetonitrile was evaporated off under reduced pressure. The micellar aqueous solution obtained was typically at a concentration of 5.2 mg·mL⁻¹ (precise determination by measuring the amount of solid after passage through the oven). The mean micellar size was 56 nm.

Coupling of the Protein (p24)

The protein was coupled to the micelles (PLA-b-P(NAS-co-NVP)) by adding a volume (typically 500 μL) of 5.2 mg/mL of micelle dispersion to the same volume of p24 in PBS, pH 7.4, at differing concentrations (0 mg·mL⁻¹ to 2.4 mg·mL⁻¹). The final coupling medium thus contained 2.6 mg·mL⁻¹ of micelles and the protein was at concentrations of 0 mg·mL⁻¹ to 1.2 mg·mL⁻¹. The samples were placed on a wheel in order to carry out stirring for 20 h at ambient temperature.

FIG. 3 illustrates a preparation of this type.

Characterization of Couplings

### SDS PAGE

The SDS-PAGE analysis showed that, for an introduced quantity of 0.12 mg of p24 per mg of copolymer, the coupling was total. When this quantity was increased (from 0.24 mg/g to 0.48 mg/g), more and more free protein was detected, indicating a “saturation” of the surface of the micelles, and thus non-quantitative coupling. The condition selected thereafter was that corresponding to a quantitative coupling, i.e. 0.12 mg of p24 per mg of copolymer, i.e. 0.3 mg/mL of p24 and 2.6 mg/mL of copolymer.

Assay of the Residual Amine Functions Remaining on the Protein after Coupling (% Modification of Amines)

90 μL of coupling medium was placed in a 96-well plate (black, NUNC) and 30 μL of 0.4 mg/mL fluorescamine (in DMSO) was added. After 20 minutes (away from the light), the fluorescence was read with a TECAN fluorimeter at an emission wavelength of 477 nm (excitation wavelength: 416 nm). The percentage of modified amines was determined by the ratio

100−(I_{fluo of coupling medium}/I_{fluo of free protein}*100).

Following coupling, the percentage of modified amines obtained was approximately 100%.

### Hydrodynamic Diameter of p24 Micelles (DLS)

The hydrodynamic diameter of the micelles, diluted by 1/50 in a 1 mM solution of NaCl, was measured by dynamic light scattering (DLS) using a ZetasizerNano S90 instrument (Malvern, UK).

The hydrodynamic diameter of the micelles was 100 nm for the PBS coupling medium control (micelles without p24); the hydrolysis of the reactive ester functions of NS to carboxylates involves deploying hydrophilic chains), and 111 nm for the micelles that had coupled to the p24.

Table 1. The low polydispersity index (PI<0.05) indicates very homogeneous sizes, whether before or after coupling. The critical micelle concentration was determined by DLS and by fluorescence by means of the hydrophobic fluorophore Nile Red, as reported previously (Handké et al. Macromol. Biosci., 13, 1213-1220 (2013)). It was of the order of 10 μg/mL, with no significant difference between the micelles and the micelles-p24.

TABLE 1
Size of reference micelles (under coupling conditions of 2.6 mg/mL
copolymer in PBS buffer but without p24) and micelles-p24.

| | Hydrodynamic | | p24 (mg/mg | CMC |
|---|---|---|---|---|
| Micelle | diameter (nm) | PI | copolymer) | (μg/mL) |
| Micelle-ref | 99.8 ± 5.5 | 0.03 ± 0.01 | — | 12 ± 4 |
| Micelle-p24 | 111.3 ± 5.1 | 0.04 ± 0.01 | 0.115 ± 0.005 | 10 ± 3 |

ELISA Protocol for the Detection of Anti-P24 Antibody

The coupled or free p24 was immobilized on the solid phase (Nunc MaxiSorp F microtitration plate) at different concentrations of PBS (cascade dilutions) for 12 hours at ambient temperature; passivation was carried out in 10% PBS—horse serum (HS); detection with a biotinylated anti-p24 antibody (rabbit) diluted in 10% PBS-Tween-HS, followed by adding streptavidin-peroxidase (horseradish peroxidase, HRP) in 10% PBS-Tween-HS 10%, and revealing with 3,3′,5,5′-tetramethylbenzidine (TMB) (absorbance at 450 nm); free p24 and single micelle controls were systematically prepared under the same conditions as those for coupling, but in the respective absence of micelles and of p24.

ELISA Results

The results obtained are represented in FIGS. 4 and 5 and demonstrate a substantial increase in the antibody detection signal compared with free p24. Furthermore, the copolymer tested in the absence of p24 at different dilutions did not involve significant background noise. This increase in sensitivity was confirmed by working with an immobilization carried out with a concentration of p24 (coupled or free) of 10 μg/mL or 1 μg/mL and varying the concentration of detection antibody.

Influence of Coupling Conditions on the Gain in Sensitivity for ELISA

The study below shows that the micellar state (i.e. nanoscale object of approximately 100 nm) was indispensable during coupling of the protein in order to increase the sensitivity of the diagnostic test (i.e. to allow the protein to become oriented towards the liquid phase by means of preferential coupling towards the end of the hydrophilic block).

### A. Coupling in Dilute/Concentrated Medium

“Concentrated” coupling: coupling of the protein under the above conditions (2.6 mg·mL⁻¹ of micelles and 0.3 mg·mL⁻¹ of p24 in PBS). As demonstrated by SDS-PAGE, coupling was total under these conditions and the micelles-p24 were 111 nm (PI=0.04) in size. The coupling medium was then diluted in PBS, in order to obtain concentrations of p24 of 10 μg·mL⁻¹ (88 μg·mL⁻¹ of micelle) or 1 μg·mL⁻¹ (8.8 μg·mL⁻¹ of micelle) for immobilization on the ELISA plate.
“Dilute” coupling: coupling of the p24 was carried out directly under the conditions used for immobilization on the ELISA plate, i.e.:
   10 μg·mL⁻¹ of p24 and 88 μg·mL⁻¹ of micelle in PBS; for this purpose, 100 μL of 0.6 mg·mL⁻¹ p24 and 100 μL of 5.2 mg·mL⁻¹ micelle were added to 5.8 mL of PBS and the solution was stirred on a rotating wheel for 20 h.
   1 μg·mL⁻¹ of p24 and 8.8 μg·mL⁻¹ of micelle in PBS; for this purpose, 10 μL of 0.6 mg·mL⁻¹ p24 and 10 μL of 5.2 mg·mL⁻¹ micelle were added to 5.98 mL of PBS and the solution was stirred on a rotating wheel for 20 h.

As can be seen in Table 2, the hydrodynamic diameters obtained for the various coupling media (direct measurement on 1 μg·mL and 10 μg·mL of p24 concentrations of coupling media) showed that the micellar state was not retained in “dilute” coupling, as indicated by the sizes of the net increase and the very high polydispersity indices, which are indicative of aggregation phenomena, in contrast to media obtained from coupling p24 under concentrated conditions. This may be explained by the fact that coupling of the p24 occurs under conditions relatively close to the CMC of the copolymer (88 μg/mL and 8.8 μg/mL of copolymer respectively for the couplings at 10 μg/mL and 1 μg/mL of p24, the CMC being 10 μg/mL). Hence, the p24, by coupling, further accentuates the capacity of the copolymer, which is already high (because of its concentration close to the CMC) to leave the micelle and form unimers. Since the micelles have been destabilized, this may be followed by a reorganization of the system with aggregation processes that are controlled to a greater or lesser extent. It should be noted that for micelles placed under the same coupling conditions but in the absence of p24, the sizes observed by DLS were 91 nm (PI=0.05) for the concentration of 88 μg/mL, and 77 nm (PI=0.3) for the concentration of 8.8 μg/mL (indicating a weakened micellar state at concentrations close to the CMC, because the standard size of the micelles of copolymer alone is 100 nm with PI=0.03, Table 1). Thus, a priori, the presence of p24 for coupling under these conditions is indeed what accentuates the micelle destabilization process.

These dilute medium coupling conditions, resulting in a “non-micellar” medium, lead to a drop in sensitivity in ELISA compared with free p24 (FIG. 6), while a significant gain was confirmed for the standard conditions (concentrated state coupling), which maintained the micellar state.

TABLE 2
Sizes obtained for various coupling media (measured
directly on coupling media with 1 μg · ml⁻¹ and
10 μg · ml⁻¹ of p24)

| Coupling | [p24] | Hydrodynamic | |
|---|---|---|---|
| type | (μg · mL⁻¹) | diameter (nm) | PI |
| Concentrated | 10 | 105 | 0.13 |
| Concentrated | 1 | 100 | 0.2 |
| Diluted | 10 | 200 | 0.5 |
| Diluted | 1 | 210 | 0.5 |

### Conclusion:

These studies show that in order to have a significant gain in sensitivity in ELISA, it is vital to carry out coupling of the protein to the copolymer organized into the form of micelles. Thus, it is necessary to react the p24 with the micelles in an aqueous medium with a copolymer concentration substantially higher than the CMC in order to preserve the micellar state.

Stability Study

The micelles-p24 (0.3 mg/mL of p24) were stored at 4° C. for 1 month. By comparison with the free p24 control, more appropriately at an optimized sensitivity (p24 from supplier stated to be 2.4 mg/mL, stored at −20° C.), the micelles-p24 retained their superiority in terms of sensitivity (FIG. 7).

### VIDAS Study on the Detection of Anti-Troponin I Antibody

The same PLA-b-P(NAS-co-NVP) copolymer as before was used to couple TnI (ITC) with a view to detecting anti-TnI antibody on an automated VIDAS® immunoassay instrument marketed by bioMérieux.

The TnI protein was coupled onto micelles of PLA-b-P(NAS-co-NVP) in PBS at a concentration of 0.137 mg/mL of TnI and 0.868 mg/mL of micelles (0.158 mg of TnI per mg of copolymer). The coupling was analyzed by SDS-PAGE gel, which showed that coupling to the micelles appeared to be almost total because free TnI was not detected. The TnI coupled thereby to the micelles was tested using VIDAS®(bioMérieux) and compared with micelles alone on the solid phase.

The automated VIDAS test was composed of 2 elements:

1—the cartridge was a plastic bar containing 10 wells sealed with an aluminum film into which the various solutions were distributed;

2—the cone, termed the SPR (Solid Phase Receptacle), acted as the pipetting system and the solid phase. Each reagent of the cartridge was aspirated then discharged via the cone. Either free TnI (ITC) in an amount of 0.03 μg/mL, or the micelles alone in an amount of 0.190 μg/mL, or the micelles-TnI in an amount of 0.03 μg/mL of TnI and 0.190 μg/mL of copolymer, in a volume of 300 μL, were immobilized on the cones.

At the end of the immobilization step, which was carried out at ambient temperature over 12 hours, the cones were emptied then brought into contact with the passivation buffer (Tris 0.2 M buffer, pH 6.2) containing a protein or peptide type saturation agent. The cones were then dried and stored at +4° C. until use.

Next, the three prepared capture phases were compared by reacting them with a tracer that was a mixture of two anti-TnI monoclonal antibodies (clones 16A11 and 7B9 marketed by HYTEST, Sweden). For practical reasons, this antibody was directly coupled to the enzyme alkaline phosphatase, in order to reduce the number of steps of the immunological reaction and reduce the duration (DEX2 protocol from VIDAS®, total duration approximately 40 min). The concentration at which this tracer was used was 0.14 μg/mL in a volume of 400 μL. The signal was generated by adding the substrate 4-methylumbelliferyl phosphate; the enzyme of the conjugate catalyzes the hydrolysis reaction of this substrate to form 4-methylumbelliferone; the fluorescence it emitted was measured at 450 nm.

Table 3 summarizes the results obtained using VIDAS® on TnI coupled to the polymer in the form of micelles and not coupled.

The signal-to-noise ratio was improved when the TnI was coupled to the polymer.

TABLE 3
VIDAS results for TnI coupled to polymer
compared with non-coupled TnI.

| | Result |
|---|---|
| SPR control | 0 |
| SPR TnI free 0.03 μg/ml | 72 |
| Signal/noise ratio | 72 |
| SPR micelles alone | 11 |
| 0.190 μg/ml of copolymer | |
| SPR micelle-TnI, | 1156 |
| 0.03 μg/ml of TnI, | |
| 0.190 μg/ml of copolymer | |
| Signal/noise ratio | 105 |

### Conclusion

Coupling of the antigen to the copolymer, organized in the form of micelles, can be used to improve the detection sensitivity.

### ELISA Study on the Detection of Anti-S100B Antibody

The aim of this study was to demonstrate the necessity of using micelles of copolymer carrying antigen in order to increase the sensitivity of diagnostic tests for immobilization of the antigen.

The antigen retained for this study was the protein S100B (native antigen, bovine brain extract, HyTest).

The polymer was PLA-b-P(NAS-co-NVP), the same as in the preceding examples. It was used either in a micellar form (in dispersion in 100% aqueous buffer), or dissolved in DMSO (“copolymer” form) for coupling with the antigen S100B in a 5% DMSO-95% aqueous buffer medium. These two protocols, implemented in parallel, were carried out in order to evaluate the influence of the coupling conditions, i.e. coupling in 100% aqueous medium with micelles versus coupling in a semi-organic DMSO/water medium with the copolymer initially dissolved in DMSO, on the gain in sensitivity of the ELISA test. The condition for protein/copolymer coupling in DMSO/aqueous buffer medium has routinely been reported in the literature (Allard et al., Bioconjugate Chem. 2001, 12, 972-979, etc.).

The polymer-antigen couplings were carried out either in solution in an Eppendorf tube or on an ELISA microplate after immobilization of the micelle or the copolymer.

Carrying Out the Couplings

A. Preparation of Micelle-S100B Conjugates

The micelles of copolymer PLA-b-P(NAS-co-NVP) were prepared as previously reported in the section “ELISA study on the detection of anti-p24 antibody”. They were initially at a concentration of 5.21 mg/mL, with a size (before coupling) of 56 nm and a PI=0.1.

The solution of S100B protein was diluted to a concentration of 0.6 mg/mL in PBS 1×.

The coupling protocol, identical to that carried out for the protein p24, is described in Table 4 below.

TABLE 4

| | | Vol, in | Vol, in | | |
|---|---|---|---|---|---|
| | Vol, | μL, of | μL, of | [S100] | [copo] |
| | PBS | 0.6 g/L S100 | micelles | (mg/mL) | (mg/mL) |
| micelles-S100 | 0 | 150 | 150 | 0.3 | 2.605 |
| micelles alone ref | 150 | 0 | 150 | 0 | 2.605 |

Incubation for 18 hours at ambient temperature.

B. Preparation of Polymer-S100B Conjugates in DMSO

The coupling protocol is described in Table 5 below.

TABLE 5

| | | Vol, in | Vol, in | Vol, in | | |
|---|---|---|---|---|---|---|
| | Vol, | μL, of | μL, of | μL, of | [S100] | [copo] |
| | PBS | 0.6 g/L S100 | DMSO | copo sol | (mg/mL) | (mg/mL) |
| copo- | 135 | 150 | 0 | 15 | 0.3 | 2.605 |
| S100 | | | | | | |
| S100 | 135 | 150 | 15 | 0 | 0.3 | 0 |
| ref | | | | | | |
| copo ref | 285 | 0 | 0 | 15 | 0 | 2.605 |

Incubation for 18 hours at ambient temperature.

Characterization of Couplings

Assay of Residual Amine Functions Remaining on Protein after Coupling (% Modification of Amines)

The assay was carried out as reported previously for the protein p24. Following coupling, the percentage of modified amines was approximately 100%.

S100B ELISA

5 different conditions, shown diagrammatically in FIG. 8, were evaluated:

1. The reference S100B immobilized directly on the microtitration plate (S100B ref);

2. The copolymers in the form of micelles alone immobilized on the plate in a first step (micelles alone ref), then plate coupling, on immobilized micelles, of the S100B protein (micelles then S100);

3. The copolymers in 5% DMSO alone, non-micellar, immobilized on the plate in a first step (copo ref) then plate coupling, onto immobilized copolymer, of the protein S100B (copolymer then S100);

4. The immobilization of micelle-S100B conjugates carried out in an Eppendorf tube (micelles+S100); and

5. The immobilization of polymer in 5% DMSO+S100B conjugates carried out in an Eppendorf tube (copolymer+S100).

### Carrying Out the ELISA

100 μL/well of single micelles (1st step condition 2) or of copolymer alone (1st step condition 3) diluted in 74 μg/mL of water were distributed into a 96-well microplate (Nunc Maxisorp F96). The microplate was incubated for 12 h at ambient temperature with stirring, in order to obtain adsorption, then emptied. In order to carry out couplings under dilute conditions, 100 μL/well of a 5 μg/mL solution of S100B was added directly to the microplate into the wells that had been incubated with the micelles alone or the copolymer alone. Furthermore, 100 μL/well of S100B in an amount of 5 μg/mL (condition 1, S100B reference) or of micelles-S100B conjugate in an amount of 74 μg/mL of micelles and 5 μg/ml of S100B (condition 4), or indeed of copolymer-S100B conjugate in an amount of 74 μg/mL of non-micellar copolymer and 5 μg/mL of S100B (condition 5) were distributed into the empty wells. The microplate was incubated for 12 additional hours at ambient temperature, with stirring, in order to obtain adsorption (conditions 1, 4, 5) or coupling (conditions 2, 3). The microplate was emptied; next, three TBS (Tris buffered saline)-Tween®-20 0.05% washes were carried out. The wells were saturated by adding 300 μL/well of passivation buffer (0.2 M Tris buffer, pH 6.2) containing a protein or peptide type saturation agent. The microplate was incubated for 1 h at 37° C., followed by 3 washes with TBS. An anti-S100B antibody (clone 8D5) in the form of Fab′ and coupled with alkaline phosphatase was distributed (100 μL/well, concentration from 0.2 μg/mL to 1.2 μg/mL), incubated for 1 hour at 37° C., followed by washing 3 times with TBS. Finally, 100 μL/well of the substrate p-nitrophenyl phosphate was added. The colorimetric signal was read at 405 nm on a microplate reader.

FIG. 9A represents the ELISA results for the various types of immobilization that were carried out. Immobilization of a micelles+S100B conjugate provided more signal than the immobilized S100B alone without increasing the background noise. The signal-to-noise ratio is thus improved when micelles are used to immobilize the protein S100B (FIG. 9B). The immobilization of polymer+S100B obtained from coupling in a DMSO/water medium, though, only induced a very low sensitivity compared with the free S100B. Finally, the fact of coupling the protein at another time to the micelles (100% aqueous buffer) or the copolymer (DMSO/aqueous buffer medium) already immobilized on the solid phase did not improve the sensitivity compared with free S100B.

### Conclusion

Coupling the antigen to the copolymer in the form of micelles can improve the sensitivity of detection compared with a free S100B system, in contrast to the same coupling on the copolymer in the non-micellar form (semi-organic DMSO/water conditions, polymer initially dissolved in DMSO), or compared with prior coupling of the antigen to a solid phase modified by the micelles or the same, but non-micellar, copolymer.

1. A method of preparing a capture phase for detecting and/or quantifying a target biological entity, said capture phase including a biological ligand for the biological entity, said biological ligand being covalently bonded to an amphiphilic polymer and being immobilized on a solid support, the method being characterized in that the biological ligand is immobilized on the solid support by bringing the solid support into contact with a dispersion of micelles formed by a plurality of chains of the amphiphilic polymer, said micelles carrying a plurality of molecules of the biological ligand on the surface thereof.

2. A preparation method according to claim 1, characterized in that the amphiphilic polymer has a hydrophobic portion oriented towards the core of the micelles and a hydrophilic portion at the surface of the micelles, the biological ligand being covalently coupled to the hydrophilic portion.

3. A method according to claim 1, characterized in that after immobilization, at least a portion of the polymer remains in the form of micelles, such that micelles formed by a plurality of amphiphilic polymer chains are immobilized at the surface of the support, said micelles carrying a plurality of molecules of the biological ligand on the surface thereof bonded with the amphiphilic polymer in a covalent manner.

4. A preparation method according to claim 1, characterized in that the immobilization is carried out in a solvent or solvent mixture constituted by at least 90% by weight, preferably at least 95% by weight, and more preferably at least 99% by weight of water.

5. A preparation method according to claim 1, characterized in that the micelles in the dispersion and/or the micelles finally immobilized on the support are formed by 100 to 5000 polymer chains and/or carry 10 to 500000 biological ligand molecules.

6. A preparation method according to claim 1, characterized in that it includes a step of covalent coupling between the biological ligand and the amphiphilic polymer, which step is carried out while the polymer is in the form of micelles, so as to form the micelles carrying a plurality of molecules of the biological ligand at the surface thereof.

7. A preparation method according to claim 6, characterized in that coupling is carried out in a solvent or solvent mixture constituted by at least 90% by weight, preferably at least 95% by weight, and more preferably at least 99% by weight of water.

8. A preparation method according to claim 5, characterized in that the coupling is carried out with a polymer concentration corresponding to at least 50 times, preferably to at least 200 times the critical micelle concentration of the polymer and/or with an amphiphilic polymer concentration at least ten times greater than that used when bringing the micelles into contact with the support.

9. A preparation method according to claim 1, characterized in that the amphiphilic polymer is a linear block polymer including at least one hydrophilic block and at least one hydrophobic block, the hydrophilic block being positioned at the surface of the micelles and carrying at least one molecule of the biological ligand by covalent bonding.

10. A preparation method according to claim 1, characterized in that the mean density of biological ligand molecules per polymer chain in the dispersion of micelles is from 0.1 to 100, and in particular from 1 to 100.

11. A preparation method according to claim 1, characterized in that the dispersion of micelles has a polydispersity index from 0 to 0.2 as determined by dynamic light scattering.

12. A preparation method according to claim 1, characterized in that the amphiphilic polymer has a molar mass greater than 5000 g/mol, preferably greater than 10000 g/mol.

13. A preparation method according to claim 1, characterized in that the amphiphilic polymer includes, or indeed is exclusively constituted by, a first linear block consisting in a hydrophobic homopolymer resulting from polymerizing a hydrophobic monomer A; and a second linear block consisting in a hydrophilic copolymer resulting from copolymerizing a monomer B carrying a reactive function X and a hydrophilic monomer C not carrying a reactive function, said second block being bonded to one end of the first block in a covalent manner.

14. A preparation method according to claim 13, characterized in that the monomer A is selected from hydrophobic derivatives of methacrylate, acrylate, acrylamide, methacrylamide, and lactides, or from styrene and its derivatives; the monomer A is preferably n-butyl acrylate, tertiobutyl acrylate, tertiobutyl acrylamide, octadecyl acrylamide, lactide, lactide-co-glycolide, or styrene.

15. A preparation method according to claim 13, characterized in that the monomer B is selected from functional derivatives of acrylate, methacrylate, acrylamide or methacrylamide, and from functional styrene derivatives; the monomer B is preferably N-acryloxy succinimide, N-methyacryloxy succinimide, 2-hydroxyethyl methacrylate, 2-aminoethyl methacrylate, 2-hydroxyethyl acrylate, 2-aminoethyl acrylate, or 1,2:3,4-di-O-isopropylidene-6-O-acryloyl-D-galactopyranose.

16. A preparation method according to claim 13, characterized in that the monomer B carries a reactive function X selected from —NH₂, —COOH, —OH, —SH, and from —C≡CH functions, ester, halogenocarboynyl, sulfhydryl, disulfide, hydrazine, hydrazone, azide, isocyanate, isothiocyanate, alkoxyamine, aldehyde, epoxy, nitrile, maleimide, halogenoalkyl, and maleimide groups, from functions that can be activated by anactivating agent such as carbodiimides, and in particular a carboxylic acid activated in the form of an ester of N-hydroxysuccinimide, pentachlorophenyl, trichlorophenyl, p-nitrophenyl, or carboxyphenyl, or indeed from bifunctional homo- or hetero-compounds.

17. A preparation method according to claim 13, characterized in that the monomer C is selected from hydrophilic derivatives of acrylamide, methacrylamide, N-vinylpyrrolidone, and oxyethylene; the monomer C is preferably N-vinylpyrrolidone or N-acryloyl morpholine.

18. A preparation method according to claim 13, characterized in that the first block has a molar mass between 1000 g/mol and 250000 g/mol.

19. A preparation method according to claim 13, characterized in that the second block has a molar mass greater than 1000 g/mol, and preferably greater than 2000 g/mol.

20. A preparation method according to claim 13, characterized in that the second block is a random copolymer with a composition, expressed as the ratio of the quantity of monomer C divided by the quantity of monomer B, the quantities being expressed in moles, which ratio is preferably in the range 1 to 10, more preferably in the range 1.5 to 4.

21. A method according to claim 1, characterized in that the biological ligand is an antigen, a hapten, or a protein.

22. A phase for capturing a target biological entity, the capture phase being characterized in that it comprises micelles immobilized on a solid support, said micelles being formed by a plurality of chains of an amphiphilic polymer, and said micelles carrying a plurality of molecules of at least one biological ligand for the target biological entity on the surface thereof, said molecules of the biological ligand being bonded to the chains of the amphiphilic polymer in a covalent manner.

23. A capture phase according to claim 22, characterized in that the micelles are immobilized on the solid support by adsorption.

24. A capture phase according to claim 23, characterized in that at least a portion of the micelles are immobilized on the solid support by adsorption by means of an interaction between the biological ligand and the solid support, a portion of the micelles optionally being immobilized on the solid support by adsorption by means of an interaction between the polymer and the solid support, the interactions involved possibly being electrostatic or ionic bonds or hydrophobic interactions, in particular.

25. A capture phase according to claim 22, characterized in that a portion of the biological ligands, corresponding in particular to at least 50% of the biological ligands present on the capture phase, is accessible and available for interacting and bonding with a target biological entity.

26. A capture phase according to claim 22, characterized in that the micelles immobilized on the support are formed by 100 to 5000 polymer chains and/or carry 10 to 500000 biological ligand molecules.

27. A capture phase according to claim 22, characterized in that the amphiphilic polymer is a linear block polymer including at least one hydrophilic block and at least one hydrophobic block, the hydrophilic block being positioned on the surface of the micelles, and carrying at least one molecule of the biological ligand by covalent bonding.

28. A capture phase according to claim 22, characterized in that the amphiphilic polymer has a molar mass greater than 5000 g/mol, preferably greater than 10000 g/mol.

29. A capture phase according to claim 22, characterized in that the amphiphilic polymer includes, or indeed is exclusively constituted by, a first linear block consisting in a hydrophobic polymer resulting from polymerizing a hydrophobic monomer A; and a second linear block consisting in a hydrophilic copolymer resulting from copolymerizing a monomer B carrying a reactive function X with a hydrophilic monomer C not carrying any reactive function, said second block being bonded to one end of the first block in a covalent manner.

30. A capture phase according to claim 26, characterized in that the amphiphilic polymer is as defined in claim 14.

31. A capture phase according to claim 22, characterized in that the biological ligand is an antigen, a hapten, or a protein.

32. A device for detecting and/or quantifying a target biological entity, the device comprising a capture phase according to claim 22, and at least one tracer for detection.

33. A device for detecting and/or quantifying a target biological entity, comprising a capture phase obtained by the method according to claim 1, and at least one tracer for detection.

34. A kit for detecting and/or quantifying a target biological entity, the kit comprising:
a solid support;
a dispersion in aqueous solution of micelles formed by chains of an amphiphilic polymer, carrying a plurality of molecules of at least one biological ligand for the target biological entity on the surface thereof, said biological ligand molecules being bonded to the chains of the amphiphilic polymer in a covalent manner; and
at least one tracer for detection.

35. A method of detecting and/or quantifying a target biological entity in vitro in a biological sample, wherein: a capture phase according to claim 22 is provided; said biological sample is brought into contact with at least the capture phase; and said target biological entity fixed on the capture phase is detected and/or quantified after the biological entity has bonded with a biological ligand molecule covalently bonded to the chains of the amphiphilic polymer of the capture phase.

36. A method of detecting and/or quantifying a target biological entity in vitro in a biological sample, wherein: a capture phase obtained by the method according to claim 1 is provided; said biological sample is brought into contact with at least the capture phase as obtained in this manner; and said target biological entity fixed on the capture phase is detected and/or quantified after the biological entity has bonded with a biological ligand molecule covalently bonded to the chains of the amphiphilic polymer of the capture phase.

37. A method of detecting and/or quantifying a target biological entity in vitro in a biological sample, wherein: a capture phase is prepared by the method according to claim 1, said biological sample is brought into contact with at least the capture phase as prepared in this way; and said target biological entity fixed on the capture phase is detected and/or quantified after the biological entity has bonded with a biological ligand molecule covalently bonded to the chains of the amphiphilic polymer of the capture phase.

38. A detection method according to claim 35, characterized in that it is a direct method in which the sample that might contain the target biological entity is brought into contact with the capture phase and bonding between the biological ligand immobilized on the support and the target biological entity is revealed by the presence of a tracer.

39. A method according to claim 38, characterized in that the tracer is a biological ligand of the target biological entity coupled to a marker.

40. A detection method according to claim 35, characterized in that it is an indirect method in which the sample that might contain the target biological entity is brought into contact with the capture phase in the presence of an analog of the target biological entity, and the bonding between the biological ligand immobilized on the support and the target biological entity is revealed by the presence of a tracer, indirectly by detecting the bonding between the biological ligand immobilized on the support and the analog of the target biological entity.

41. A method according to claim 40, characterized in that the tracer is the analog of the target biological entity coupled to a marker.

42. A method according to claim 39, characterized in that the marker is selected from enzymes, chromophores, radioactive molecules, fluorescent molecules and electrochemiluminescent salts.

43. A method according to claim 41, characterized in that the marker is selected from enzymes, chromophores, radioactive molecules, fluorescent molecules and electrochemiluminescent salts.
